(19) United States
(12) Patent Application Publication
(10) Pub. No.: US 2026/0240464 A1
(43) Pub. Date: Aug. 20, 2026

(54) COMPOSITION FOR BLOOD GLUCOSE MEASUREMENT AND ELECTROCHEMICAL SENSOR USING SAME

(71) Applicant: SEOUL NATIONAL UNIVERSITY R&DB FOUNDATION, Seoul (KR)

(72) Inventors: YOUNG BIN CHOY, Seoul (KR); JAE HOON HAN, Seoul (KR); CHO RIM KIM, Gyeonggi-do (KR)

(21) Appl. No.: 19/469,527

(22) PCT Filed: Sep. 19, 2023
(86) PCT No.: PCT/KR2023/014149

§ 371 Date: Sep. 26, 2025

(30) Foreign Application Priority Data
Mar. 28, 2023 (KR) 10-2023-0040373

(51) Int. Cl. A61B 5/1473; A61B 5/00; A61B 5/145; C08F 8/12; C08F 8/42; C08K 3/04; C09D 5/24; C09D 7/61; C09D 153/00

(52) U.S. Cl. CPC A61B 5/14735; A61B 5/14532; A61B 5/685; C08F 8/12; C08F 8/42; C09D 5/24; C09D 7/61; C09D 153/005; C08F 2800/10; C08K 3/041; C08K 2201/001; C08K 2201/011

(57) ABSTRACT

A composition for blood glucose measurement includes a copolymer including three types of monomers having a hydrophobic group, a hydrophilic group, and a boronic acid moiety, and a conductive material mixed with the copolymer, thereby enabling volume changes in response to glucose to be converted into electrical signals. An electrochemical sensor for blood glucose measurement includes a working electrode that includes the composition.

### BACKGROUND

### 1. Technical Field

The present invention relates to a composition for blood glucose measurement and an electrochemical sensor using the same, which may be utilized in the medical field.

### 2. Background Art

Diabetes is a typical chronic disease caused by insufficient secretion or improper functioning of insulin, and patients with diabetes typically exhibit elevated blood glucose levels compared to healthy individuals. In order to diagnose and treat diabetes, continuous blood glucose monitoring is required, and diabetic patients often monitor their blood glucose levels several times a day by pricking their fingertips with a needle, which induces pain and discomfort. However, in conventional methods, diabetic patients must measure their blood glucose levels through self-blood collection each time, which not only causes repeated pain but also increases the risk of calluses, pigmentation, and secondary infection. In addition, it is difficult to comply with correct blood sampling procedures (e.g., squeezing the fingertip, exposure to external contaminants), which leads to reduced measurement accuracy.

In this regard, microneedles have been recently developed as promising tools for measuring glucose concentration in interstitial fluid (ISF) with minimal invasiveness and without causing pain. However, since most microneedle-based sensors utilize glucose oxidase, their operational lifetime is limited due to the inherent instability of the enzyme. Therefore, there is a need for the development of a novel blood glucose measurement method that is improved in various aspects, does not use enzymes, highly accurate, suitable for continuous monitoring, and applicable in a minimally invasive manner.

### SUMMARY

It is an object of the present invention to provide a composition for blood glucose measurement.

It is another object of the present invention to provide an electrochemical sensor for blood glucose measurement that utilizes the composition.
1. A composition for blood glucose measurement, including: a copolymer including a hydrophobic first monomer, a hydrophilic second monomer, and a third monomer having a boronic acid moiety bonded thereto; and a conductive material mixed with the copolymer.
2. The composition for blood glucose measurement according to the above 1, wherein the molar ratio of the first monomer, the second monomer, and the third monomer of the copolymer is 1:0.7 to 0.9:0.1 to 0.3.
3. The composition for blood glucose measurement according to the above 1, wherein the conductive material is included in an amount of 0.7 to 1.5 wt % based on the total weight of the copolymer and the conductive material.
4. The composition for blood glucose measurement according to the above 1, wherein the first to third monomers are respectively represented by Formulas 1 to 3 below:

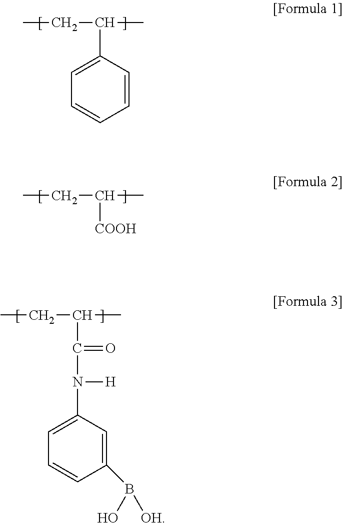

5. An electrochemical sensor for blood glucose measurement including a working electrode that includes the composition for blood glucose measurement according to any one of the above 1 to 4.
6. The electrochemical sensor for blood glucose measurement according to the above 5, further including a counter electrode, a reference electrode, a measuring part configured to measure a current flowing between the working electrode and the counter electrode, and a power supply part configured to apply a voltage to the electrodes.
7. The electrochemical sensor for blood glucose measurement according to the above 5, wherein the working electrode is at least partially formed of the composition, or is at least partially coated with the composition.
8. The electrochemical sensor for blood glucose measurement according to the above 5, wherein the working electrode is in the form of a microneedle.

The composition of the present invention undergoes swelling and shrinkage in response to glucose concentration, and this physical response may be converted into a change in electrical conductivity. Therefore, blood glucose levels can be measured with high sensitivity by detecting the corresponding electrical signal. The copolymer included in the composition of the present invention includes monomers having a hydrophobic group, a hydrophilic group, and a boronic acid moiety, and is capable of effectively absorbing body fluids to provide a composition suitable for blood glucose measurement. Since the composition of the present invention utilizes a reversible interaction with glucose, it can detect both increasing and decreasing continuous changes in glucose concentration, enabling real-time blood glucose monitoring.

The present invention also provides an electrochemical sensor for blood glucose measurement that employs the above-described composition, and exhibits the above-described advantages of the composition. In addition, the composition may be applied in various forms, as needed, by being partially or entirely incorporated into an electrode that reacts with glucose in the sensor, or by being coated on a portion or the entire surface of the electrode. The electrode may be configured in the form of a microneedle, thereby enabling effective blood glucose measurement while minimizing patient discomfort in a minimally invasive manner.

### BRIEF DESCRIPTION OF THE DRAWINGS

FIG. 1 is a schematic diagram illustrating the swelling and shrinkage response of a microneedle sensor coated with the composition of the present invention, depending on glucose concentration.

FIGS. 2A and 2B illustrate a manufacturing process of a microneedle sensor coated with the composition of the present invention and an example of the manufactured sensor.

FIGS. 3A and 3B illustrate microneedles manufactured by coating the composition of the present invention for different durations and the corresponding changes in measured current.

FIG. 4 is a schematic diagram illustrating the swelling and dehydration response of a microneedle sensor formed of the composition of the present invention, depending on glucose concentration, in one exemplary embodiment.

FIG. 5 illustrates a manufacturing process of a microneedle sensor formed of the composition of the present invention and a resulting sensor.

FIG. 6 is a set of graphs illustrating changes in the sensing sensitivity of the microneedles according to the ratio of the copolymer in the composition of the present invention.

FIG. 7 is a set of graphs illustrating changes in the sensing sensitivity of the microneedles according to the loading concentration of a conductive material in the composition of the present invention.

FIGS. 8A to 8C are graphs illustrating in vivo experimental results according to an embodiment of the present invention. Specifically, FIG. 8A shows results from an experiment conducted on healthy mice, FIG. 8B shows results from an experiment conducted on type 1 diabetic (T1D) mice, and FIG. 8C shows results of continuous blood glucose monitoring for 12 hours using an MN sensor in healthy mice.

FIGS. 9A and 9B are images obtained by applying the MN sensor of the present invention to the surface of mouse skin and performing H&E staining to confirm biosafety.

### DETAILED DESCRIPTION

Hereinafter, the present invention will be described in detail.

The present invention relates to a composition for measuring blood glucose, which includes a copolymer including a hydrophobic first monomer, a hydrophilic second monomer, and a third monomer having a boronic acid moiety bonded thereto; and a conductive material mixed with the copolymer.

The composition of the present invention undergoes swelling and shrinkage in response to glucose concentration, and this physical response may be converted into a change in electrical conductivity.

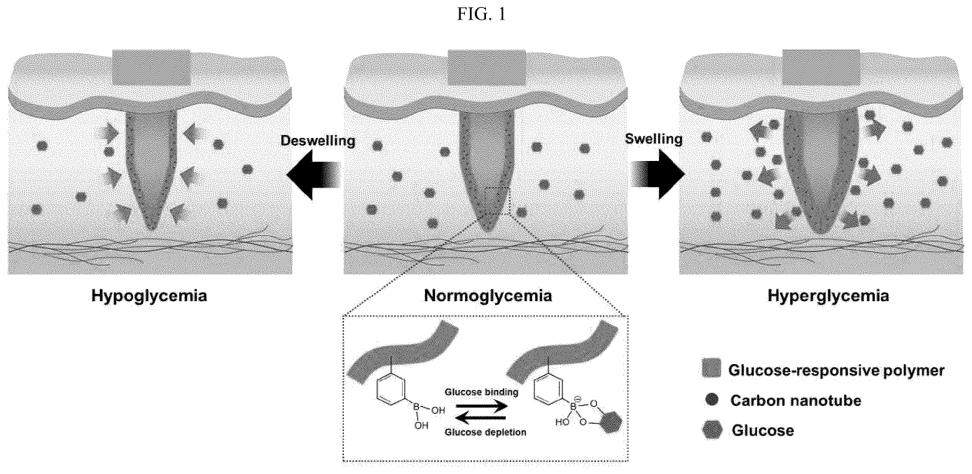

Specifically, the boronic acid moiety bonded to the third monomer in the copolymer of the present invention reacts with glucose to form a glucose-boronic acid ester via its diol group, as illustrated in the reaction scheme shown at the bottom of FIG. 1 of the present disclosure. Through the combination of monomers bearing a hydrophobic group, a hydrophilic group, and a boronic acid moiety, the copolymer of the composition may effectively absorb body fluids, and through the above reaction, increase its volume in proportion to the blood glucose concentration.

The composition of the present invention, through the mixing of the copolymer and the conductive material, may convert an increase or decrease in the volume of the copolymer into a corresponding decrease or increase in the density of the conductive material. As a result, the conductivity of the composition may be decreased or increased accordingly. Thus, for example, the blood glucose level of a subject may be measured by detecting a change in the resistance of the composition, or a change in current resulting therefrom.

The reaction between the boronic acid and glucose is reversible and may be repeatedly and continuously utilized in environments where the blood glucose level fluctuates, including both increases and decreases.

Since the first monomer and the second monomer in the composition of the present invention do not directly react with glucose, their types are not particularly limited. The first monomer and the second monomer may be selected by those skilled in the art as copolymerizable monomers having hydrophobicity or hydrophilicity, respectively.

For example, the first monomer may include a carbon chain substituted with a hydrophobic functional group. The carbon chain may have 1 to 3 carbon atoms (C1 to C3). The hydrophobic functional group may be, for example, a straight or branched C1 to C20, C1 to C10, C5 to C20, or C5 to C10 alkyl group, or C6 to C20 or C6 to C12 aryl group.

For example, the second monomer may include a carbon chain substituted with a hydrophilic functional group. The carbon chain may have 1 to 3 carbon atoms (C1 to C3). The hydrophilic functional group may be, for example, a hydroxyl group, a carboxyl group, a carbonyl group, an ether group, an ester group, an aldehyde group, a sulfonic acid group, or an amino group.

The degree of hydrophilicity of the copolymer may be adjusted by varying the combination ratio of the first to third monomers. This allows for appropriate body fluid inflow and the degree of swelling required for the reaction with glucose.

For example, in the composition of the present invention, the molar ratio of the first monomer, the second monomer, and the third monomer in the copolymer may be 1:0.5 to 2:0.1 to 2, 1:0.5 to 1.5:0.1 to 1, or 1:0.5 to 1:0.1 to 0.5,

Specifically, the molar ratio may be 1:0.7 to 0.9:0.1 to 0.3, 1:0.7 to 0.85:0.1 to 0.3, or 1:0.8:0.15 to 0.25. As a result, the glucose sensitivity of the composition may be particularly increased.

Since the third monomer is intended to introduce a boronic acid moiety, which functions as the glucose-responsive site, into the polymer, any structure having a boronic acid moiety may be used without particular limitation, as long as it does not interfere with the reaction with glucose. For example, the boronic acid moiety may be bonded through a functional group other than the diol portion that forms a covalent bond with glucose.

As the boronic acid, for example, phenylboronic acid (PBA), aminophenylboronic acid (APBA), specifically 3-aminophenylboronic acid (3-APBA), 4-mercaptophenylboronic acid (4-MPBA), 4-formylphenylboronic acid (4-FPBA), 4-(methylcarbamoyl)phenylboronic acid (4-MCPBA), 2-formylphenylboronic acid (2-FPBA), or 2-((dimethylamino)methyl)phenylboronic acid may be used, but is not limited thereto.

For example, since the aminophenylboronic acid (APBA) includes an amino group, it may form a covalent bond with a structure having a carboxyl group, such as through an amide bond.

Specifically, in the embodiment of the present disclosure, a monomer of polystyrene as a representative hydrophobic polymer, a monomer of polyacrylic acid as a hydrophilic polymer, and 3-APBA bound to polyacrylic acid were respectively used as the first to third monomers, and these may be represented by Formulas 1 to 3 below.

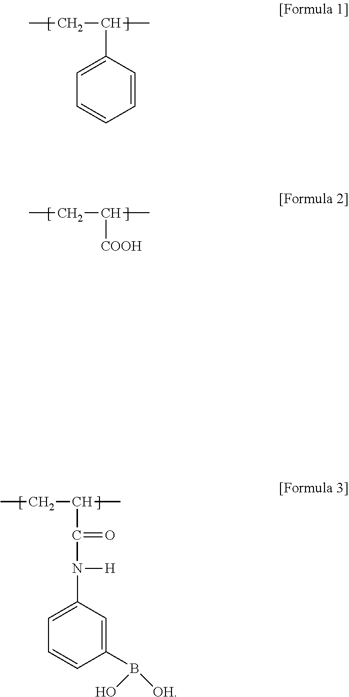

The conductive material is mixed with the copolymer to impart a change in electrical conductivity. The conductive material may be selected from the group consisting of, for example, carbon nanotubes (CNTs), graphene, carbon black, acetylene black, and Ketjen black.

The amount of the conductive material mixed may be, for example, 0.1 to 10%, 0.1 to 5%, 0.5 to 5%, and preferably 0.7 to 5%, 0.7 to 3%, 0.7 to 2%, and 0.7 to 1.5%, based on the total weight of the copolymer and the conductive material. As a result, the glucose sensitivity of the composition may be particularly enhanced.

The present invention also provides an electrochemical sensor for measuring blood glucose, which includes the composition included in working electrodes.

The composition is included in the working electrodes, in which the target reaction of the sensor occurs, because it responds to the glucose concentration to be measured and exhibits changes in volume and electrical conductivity. Since the specific configuration, principle of blood glucose measurement, and effects have already been described above, further description will be omitted to avoid redundancy.

The electrode may be at least partially formed of the composition, or may be at least partially coated with the composition.

For example, the electrode may be manufactured by molding the composition into an electrode shape as needed, or by coating the electrode with the composition using a conventional method such as electrospray coating, dip coating, spray coating, roller coating, or flow coating.

The sensor and its configuration may vary depending on the intended use by those skilled in the art.

For example, the electrode may be manufactured in the form of a microneedle, which enables blood glucose measurement in a simple manner with minimal invasiveness to the patient.

The microneedles may have various shapes and numbers, such as square pyramids or cones, and are not particularly limited.

Specifically, the sensor may include counter electrodes, reference electrodes, a measuring part configured to measure a current flowing between the working electrode and the counter electrode, and a power supply part configured to apply a voltage to the electrodes.

The voltage applied from the sensor may be appropriately selected by those skilled in the art in consideration of in vivo use, and may be, for example, greater than 0 V and less than or equal to 1 V, or 0.1 V to 0.6 V relative to the reference electrode, but is not limited thereto.

The sensor may include a conversion unit configured to convert the measured current into a blood glucose level as needed, and a display unit configured to display the measurement results.

The sensor may be used for applications attached to or inserted into the body.

The sensor may be included as a part of various medical devices requiring blood glucose sensing, depending on the needs of those skilled in the art.

Hereinafter, the present invention will be described in detail through examples. However, the following examples are provided for illustrative purposes only to facilitate understanding of the present invention, and are not intended to limit the scope of the present invention in any way.

### Example

### 1. Preparation of Polymer Solution

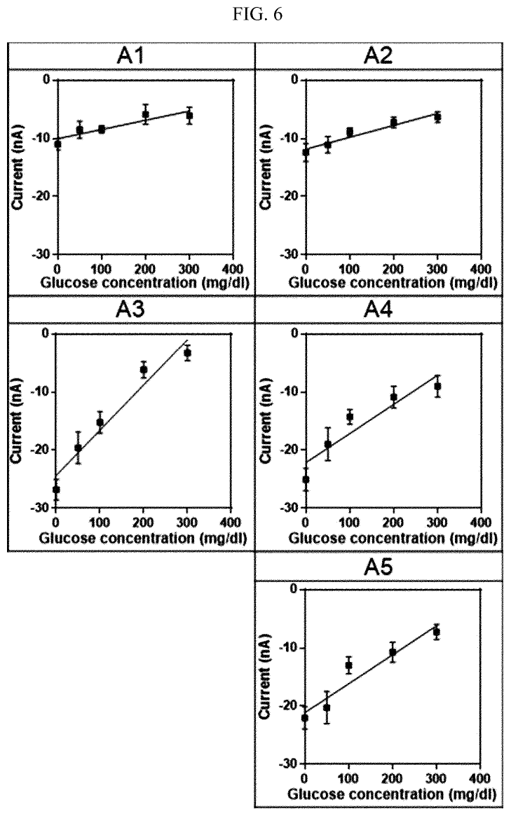

A glucose-reactive polymer was copolymerized using a radical initiator under an inert atmosphere, dialyzed for one week, and then freeze-dried to obtain the final product. Specifically, styrene and tert-butyl acrylate were copolymerized using a radical initiator under an inert atmosphere, and the resulting copolymer was hydrolyzed with trifluoroacetic acid to produce polystyrene-block-poly(acrylic acid) (hereinafter referred to as “PS-b-PAA”). PS-b-PAA and 3-aminophenylboronic acid (APBA) were dissolved in DMF and allowed to react for 24 hours. In this experiment, the molar ratios of styrene, tert-butyl acrylate, and 3-aminophenylboronic acid were adjusted according to the conditions A1 to A5 shown in Table 1 below. The slope shown at the bottom of the table refers to the slope in FIG. 6, which presents the experimental results described below.

TABLE 1

| Name | A1 | A2 | A3 | A4 | A5 |
|---|---|---|---|---|---|
| Molar ratio | 1:0.95:0.05 | 1:0.8:0.1 | 1:0.8:0.2 | 1:0. [symbol] :0.36 | 1:0.6:0.4 |
| Weight ratio (wt %) | 57:38:5 | 55:35:10 | 52:29:19 | 48:22:30 | 47:1 [symbol] :34 |
| Slope(nA/mg/dl) | 0.01578 | 0.02043 | 0.077 [symbol] 2 | 0.04992 | 0.04971 |

[symbol]

indicates data missing or illegible when filed

The synthesized PS-b-P(AA-co-AAPBA) copolymer was dissolved in N-methyl-2-pyrrolidone containing dispersed CNTs.

### 2. Manufacture of Microneedles

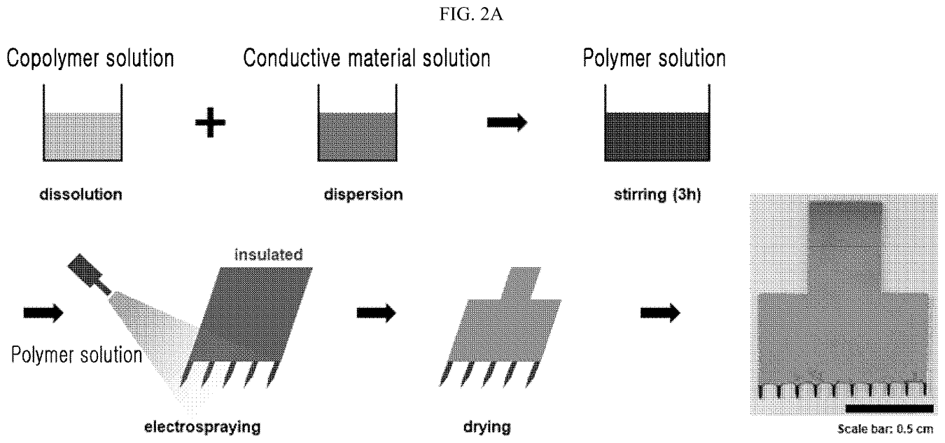

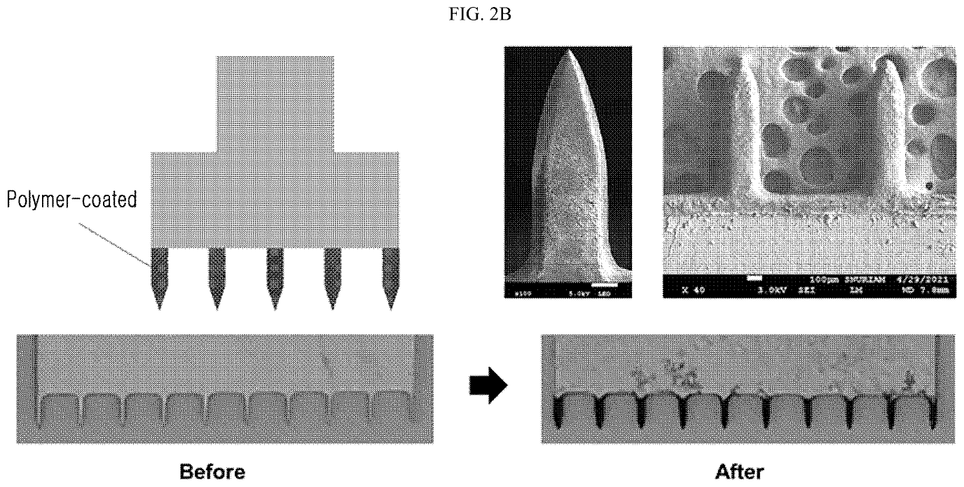

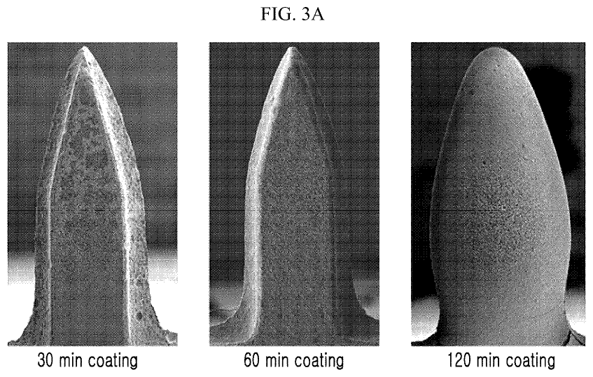

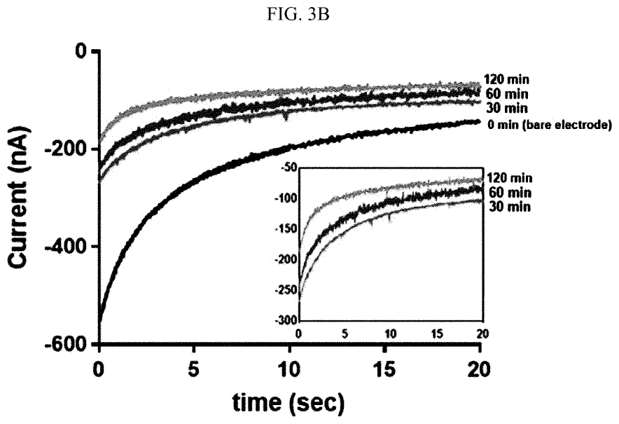

As shown in FIG. 2A, microneedles were manufactured by a method of coating with the prepared solution. First, a stainless steel substrate having a thickness of 200 μm (316 L grade) was patterned to a microneedle length of 1000 μm through etching. The prepared polymer solution was then electrosprayed onto the tips of the microneedles. FIG. 2B shows SEM images of the manufactured microneedles. In addition, it was confirmed that the thickness of the coating on the microneedle varied by varying the spraying time, and thus the current value measured in the same solvent could be adjusted (FIG. 3).

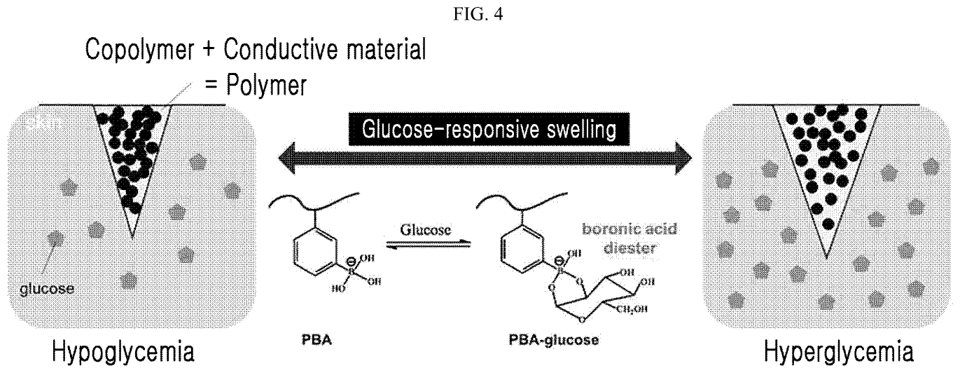

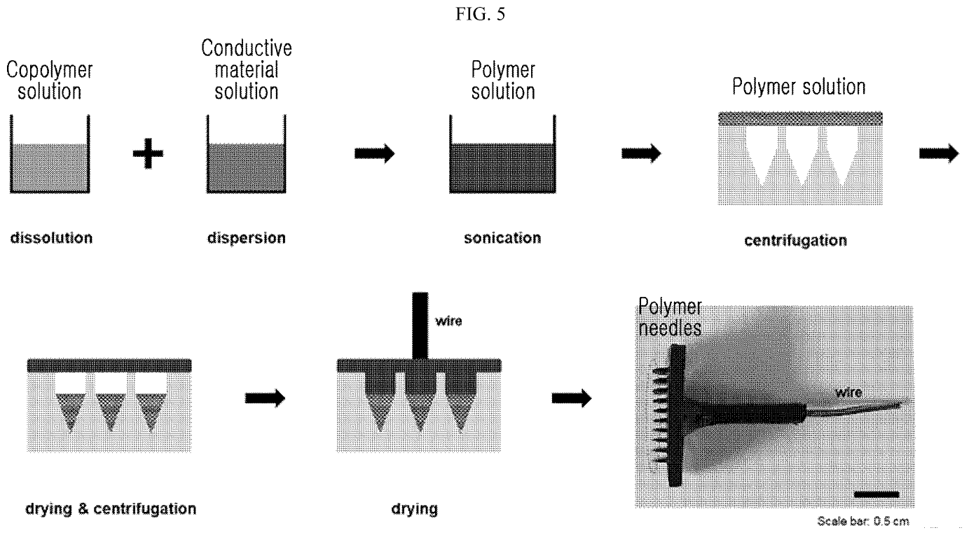

Further, as shown in FIGS. 4 and 5, microneedles were also manufactured by filling a mold with the prepared polymer solution and casting the solution. After molding the polymer solution, the back side of the mold was filled with a highly conductive polymer so that wires could be formed thereon, and then the resulting structure was dried.

### 3. In Vitro Experiment of Microneedle Sensor

The microneedle sensor of the present invention includes three microneedle electrodes (a working electrode, a counter electrode, and a reference electrode). The microneedle coated with the polymer solution was used as the working electrode, and the microneedle tip of the reference electrode was coated with Ag/AgCl ink, and the microneedle tip of the counter electrode was sputtered with Pt. The three electrodes were embedded in a PDMS mold to form a microneedle sensor.

In addition, for the in vitro evaluation of the manufactured sensor, agarose gels with various glucose concentrations were prepared. The microneedle sensor was sequentially applied to the agarose gels of each glucose concentration, and the current measurement response was recorded using a standard three-electrode electrochemical potentiostat. As shown in Table 1 and FIG. 6, the current measurement graph according to the change in glucose concentration was obtained through an experiment in which the ratio of the copolymer monomers was adjusted, and the slope of the graph was particularly high under the A3 condition, confirming that sensing with high sensitivity was possible.

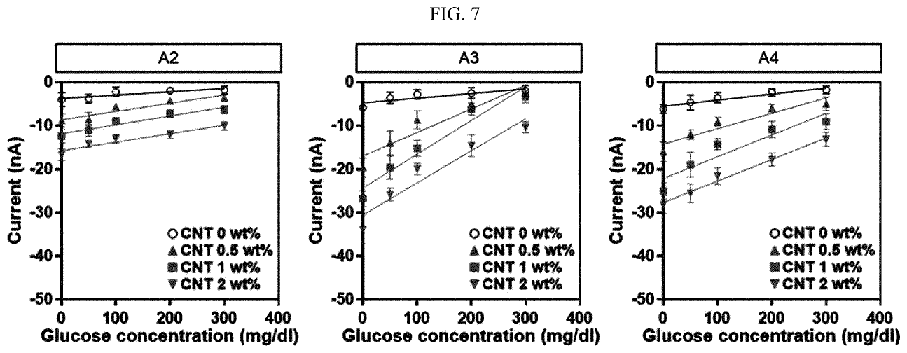

FIG. 7 additionally shows the experimental results with different weight ratios of the conductive material, and the conditions and results are shown in Table 2 below and FIG. 7. It was confirmed that in all of the A2, A3 and A4 conditions used, particularly high sensitivity was achieved when CNTs were used at 1 wt %.

TABLE 2

| Name | | | A2 | A3 | A4 |
|---|---|---|---|---|---|
| Molar ratio | | | 1:09:0.1 | 1:0.8:0.2 | 1:0.65:0.35 |
| Slope | 0 | wt % | 0.00764 | 0.01072 | 0.01 [symbol] 97 |
| (nA/mg/dl) | 0. [symbol] | wt % | 0.01 [symbol] | 0.06436 | 0.03551 |
| | 1 | wt % | 0.02043 | 0.077 [symbol] 2 | 0.04992 |
| | 2 | wt % | 0.01 [symbol] 59 | 0.07360 | 0.04943 |

[symbol]

indicates data missing or illegible when filed

### 4. In Vivo Experiment Using a Mouse Model

The wearable microneedle sensor (hereinafter referred to as an “MN sensor”) manufactured and coated under the above-described A3 condition with 1 wt % CNT was applied to the dorsal skin of a shaved mouse model to evaluate its effectiveness.

### 4.1. Analysis of Accuracy of Blood Glucose Measurement

Specifically, in order to confirm the blood glucose measurement performance in vivo, the MN sensor was applied to both healthy mice and type 1 diabetic (T1D) mice, and the glucose level was estimated using Equation 1 below.

$$Glucose concentration ( mg / dl ) = 12.9 ( mg / dl / nA ) \times Measured current value ( nA ) + 312.9 ( mg / dl ) \qquad [ Equation 1 ]$$

At the same time, the blood glucose level was measured using the blood glucose meter as a reference value. The T1D mice were injected intraperitoneally with a streptozotocin solution (60 mg/kg) prepared in 0.09 M citrate buffer (pH 4.0) to destroy pancreatic cells, and 3 days later, the mice were fasted overnight, and only those with a blood glucose level of 300 mg/dl or higher were used in the experiment. A hyperglycemic state was induced in healthy mice by injecting glucagon (100 μg/kg), which caused a transient increase in blood glucose levels over 2 hours. In the case of T1D mice, an insulin solution (2.0 U/kg) was injected to decrease the blood glucose level under the hyperglycemic condition.

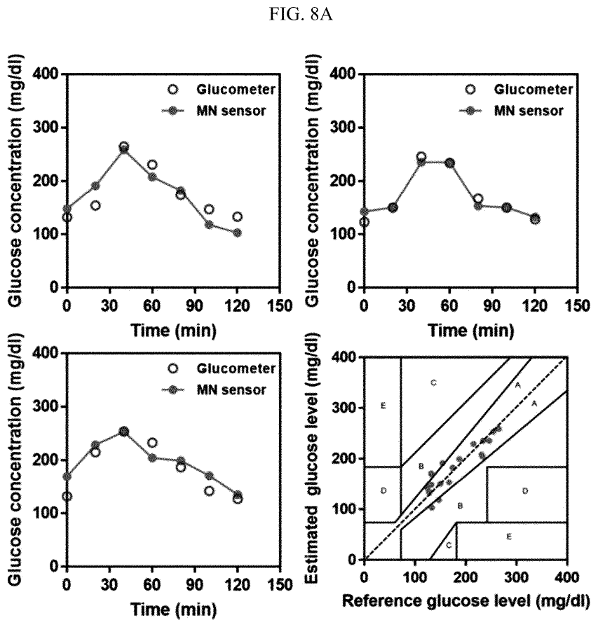

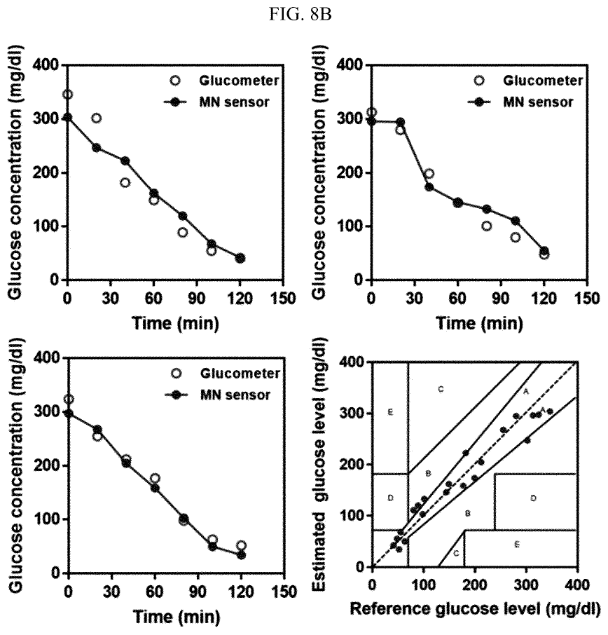

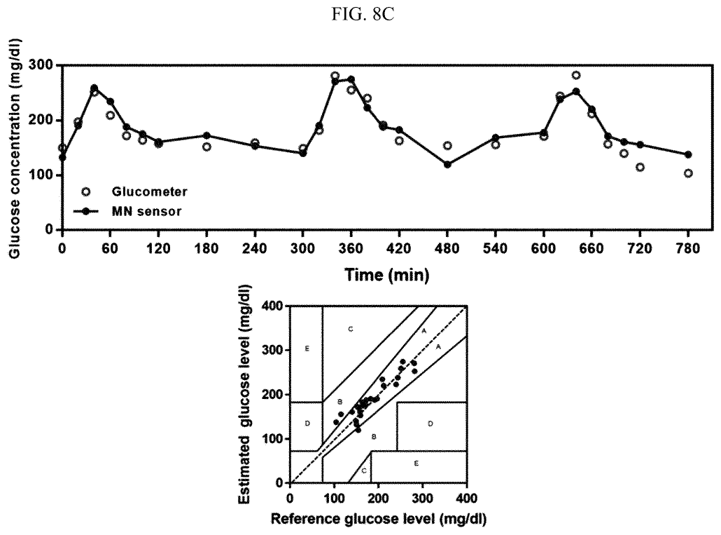

As shown in FIGS. 8A and 8B, the blood glucose levels estimated using the MN sensor were consistent with the reference values measured using the blood glucose meter for all test animals. Each graph shows the results of experiments on three animals, and on the far right, the measured data points were plotted on a Clarke error grid, confirming that all points fell within regions A and B. FIG. 8A shows the results for healthy mice, and FIG. 8B shows the results for T1D mice. In addition, 81% and 76% of the measured data points for the healthy mice and T1D mice, respectively, were located within region A of the Clarke error grid. This shows that the MN sensor of the present invention has clinically acceptable accuracy compared to the blood glucose meter, which is a clinically approved method for measuring blood glucose.

### 4.2. Analysis of Continuous Reversible Blood Glucose Monitoring

To verify whether continuous and reversible blood glucose monitoring is possible, the MN sensor of the present invention was worn for 12 hours and then replaced with another sensor for the next 12 hours, and monitoring was performed throughout a 24-hour period. To evaluate the validity of the experiment, the inventors simulated three meals a day with three injections of glucagon while applying the MN sensor to healthy mice for 12 hours.

The results are shown in FIG. 8C, and it can be confirmed that the estimated blood glucose level is consistent with the reference value, and all data points fell within regions A and B of the Clarke error grid. In addition, 88% of the data points were located in region A, indicating that the MN sensor has high accuracy for continuous blood glucose monitoring.

### 4.3. In Vivo Safety Analysis

In addition, the in vivo safety of the MN sensor of the present invention was verified through biopsy analysis of H&E-stained tissues. Specifically, after applying the MN sensor, skin tissue was biopsied, fixed with a formalin solution, and embedded in paraffin to create a block. The block was cut into 4 μm-thick sections, mounted on a glass slide, and stained with hematoxylin and eosin (H&E). The tissue slide images were obtained at 4× magnification using an optical microscope (IX53, Olympus, Japan) and evaluated by a professional pathologist.

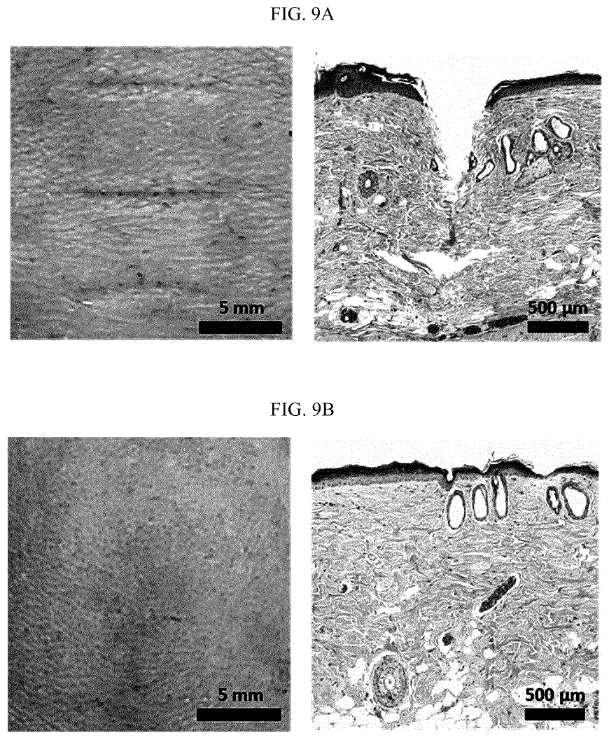

As shown in FIG. 9A, the destruction of the stratum corneum was clearly observed, indicating that the MN sensor was properly inserted into the dermis layer of the skin. In addition, when observed 9 hours after the removal of the MN sensor, the results in FIG. 9B confirmed that the skin had completely recovered. The H&E-stained images showed no significant infiltration of inflammatory cells in the tissue, confirming the in vivo safety of the MN sensor.

1: A composition for blood glucose measurement, comprising:
a copolymer comprising a hydrophobic first monomer, a hydrophilic second monomer, and a third monomer having a boronic acid moiety bonded thereto; and
a conductive material mixed with the copolymer.

2: The composition for according to claim 1, wherein the molar ratio of the first monomer, the second monomer, and the third monomer of the copolymer is 1:0.7 to 0.9:0.1 to 0.3.

3: The composition for according to claim 1, wherein the conductive material is included in an amount of 0.7 to 1.5 wt % based on the total weight of the copolymer and the conductive material.

4: The composition for according to claim 1, wherein the first to third monomers are respectively represented by Formulas 1 to 3:

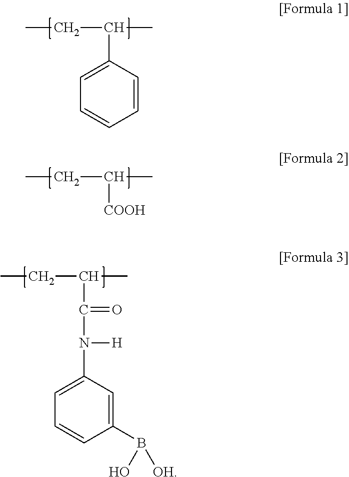

5: An electrochemical sensor for blood glucose measurement, comprising a working electrode comprising the composition for blood glucose measurement according to claim 1.

6: The electrochemical sensor f according to claim 5, further comprising a counter electrode, a reference electrode, a measuring part configured to measure a current flowing between the working electrode and the counter electrode, and a power supply part configured to apply a voltage to the electrodes.

7: The electrochemical sensor according to claim 5, wherein the working electrode is at least partially formed of the composition, or is at least partially coated with the composition.

8: The electrochemical sensor according to claim 5, wherein the working electrode is in the form of a microneedle.
